(19) United States
(12) Patent Application Publication
(10) Pub. No.: US 2006/0259009 A1
(43) Pub. Date: Nov. 16, 2006

(54) Guidewire loader for bifurcated vessel

(75) Inventor: Robert J. Murray, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/128,610

(22) Filed: May 12, 2005

(51) Int. Cl. A61M 25/00

(52) U.S. Cl. 604/523

(57) ABSTRACT

A guidewire loader catheter for delivering a guidewire across a bifurcation in a blood vessel, including a method of use, is provided. The guidewire loader catheter includes a catheter shaft having a first tube, with a first lumen extending therethrough, a second tube, with a second lumen extending therethrough, and a torque means for controlling the catheter shaft. The second tube has a longitudinal length generally less than the longitudinal length of the first tube. A main vessel guidewire is inserted through the first lumen of the first tube and then a branching vessel guidewire is inserted through the second lumen of the second tube to be delivered across the bifurcation.

### BACKGROUND OF THE INVENTION

1. Field of the Invention

The present invention relates to medical catheters. More specifically, the invention relates to a catheter used to deliver a guidewire across a bifurcated blood vessel.

2. Related Art

Cardiovascular disease, including atherosclerosis, is a leading cause of death in the United States. As a result, many procedures have been developed to treat and diagnose various conditions that arise from cardiovascular disease, such as percutaneous transluminal coronary angioplasty, commonly referred to as “angioplasty” or “PTCA”, implantation of vascular prostheses or stents, delivery of therapeutic substances (such as anti-vaso-occlusion agents or tumor treatment drugs), and delivery of radiopaque agents for radiographic viewing. At times, multiple prostheses must be delivered to a bifurcated lesion across branching blood vessels. Various techniques have been used to deliver multiple prostheses in order to provide radial support to both a main blood vessel and a branching blood vessel.

The first step in treating a bifurcated lesion is getting guidewires into the branching blood vessels. This step is often the most difficult and time consuming step in the procedure, and will often result in wire twisting. When a bifurcated lesion is being treated with a single device, that utilizes two wires, having the wires twisted will prohibit the physician from being able to track the device to the corina, or fork, of the bifurcation. Overall, wire twisting will make a procedure more complicated and time consuming.

There is a need for a simple, low profile device, which can be used to deliver multiple guidewires across a bifurcation, while preventing wire twisting.

### BRIEF SUMMARY OF THE INVENTION

In accordance with one embodiment of the present invention, there is provided a guidewire loader catheter for delivery of a guidewire across a bifurcation in a blood vessel. The guidewire loader catheter includes a catheter shaft having a first tube, with a first lumen extending therethrough, and a second tube, with a second lumen extending therethrough. The longitudinal length of the second tube is less than the longitudinal length of the first tube. The second tube is tangentially attached to the first tube such that the second lumen runs parallel to the first lumen, and the first tube extends distally beyond the second tube. The guidewire loader catheter further includes a torque means for controlling the catheter shaft when the catheter shaft is inserted into a main blood vessel, such that the second lumen can be positioned to deliver a guidewire to a branching blood vessel. The torque means may include a torquable handle attached to a proximal section of the catheter shaft. An embodiment of the present invention includes a surrounding sheath, formed of a polymer material having metallic braids imbedded therein, that envelopes the first and second tubes of the catheter shaft. The first and second tubes may be formed of PEBAX, polyethylene, PEEK, nylon, nylon derivatives, or polyimides. In an alternative embodiment, the catheter shaft is formed from a polymer material having metallic braids imbedded therein.

In accordance with another embodiment of the present invention, there is provided a dual-lumen guidewire loader catheter having a tubular shaft formed of a polymer material and a torquable handle attached to a proximal section of the tubular shaft to control the rotational position of the tubular shaft within a blood vessel. The tubular shaft includes a first lumen extending from the proximal end of the tubular shaft to the distal end of the tubular shaft. The tubular shaft also includes a second lumen, extending from the proximal end of the tubular shaft to a point short of the distal end of the tubular shaft. The second lumen is parallel to the first lumen, and the second lumen has a longitudinal length less than the longitudinal length of the first lumen. As such when placed within a patient's vasculature, the tubular shaft is configured to deliver a first guidewire to a first location in a blood vessel, through the first lumen, and a second guidewire to a second location, in a bifurcated blood vessel, through the second lumen. In one embodiment, the tubular shaft has a midsection having a cross-section of a generally circular shape. In an alternative embodiment, the cross-section has a generally oval shape. In yet another embodiment, the cross-section has a generally shotgun-barrel shape (FIG. 8).

In accordance with another embodiment of the present invention, there is provided a guidewire loader catheter having a first catheter shaft, a second catheter shaft, and a guide member slidably coupled to the first catheter shaft. The first catheter shaft includes a branch guidewire lumen extending longitudinally through the first catheter shaft. The first catheter shaft further includes a guideway extending longitudinally along a length of a proximal portion of the first catheter shaft and radially from the branch guidewire lumen to an outer surface of the proximal portion of the first catheter shaft. The second catheter shaft includes a main guidewire lumen extending longitudinally through the second catheter shaft. The second catheter shaft extends from a distal portion of the first catheter such that a proximal end of the second catheter shaft is proximal of a distal end of the first catheter shaft, a distal end of the second catheter shaft is distal of a distal end of the first catheter shaft, and the main guidewire lumen runs parallel to the branch guidewire lumen. The guide member is configured to provide access to the branch guidewire lumen via the guideway. The guidewire loader catheter further includes a torquable handle disposed about the proximal portion of the first catheter shaft, proximal to the guide member. In one embodiment, the guide member has a main guidewire lumen, aligned with the main guidewire lumen of the second catheter shaft, such that a guidewire may be inserted through the main guidewire lumen of the second catheter shaft and through the main guidewire lumen of the guide member. The guidewire loader catheter may be formed from PEBAX, polyethylene, PEEK, nylon, nylon derivatives, or polyimides.

In accordance with another embodiment of the present invention, there is provided a method of delivering multiple guidewires across a bifurcation. The method includes the steps of providing a guidewire loader catheter in accordance with the present invention, and inserting a first guidewire into and through a first blood vessel, to a point distal of a bifurcation between the first blood vessel and a branching blood vessel. Next, the guidewire loader catheter is tracked over the first guidewire, such that the guidewire is disposed within a first lumen of a first tube, and the guidewire loader catheter is advanced until a distal end of the first tube extends distally beyond the bifurcation and a distal end of the second tube remains proximal of the bifurcation. The method further includes adjusting the rotational position of the guidewire loader catheter such that the distal end of the second tube is aligned with respect to the branching blood vessel. Such adjusting step is accomplished by using a torquable handle, or alternative torque means, to rotate the guidewire loader catheter within the blood vessel. Such adjustment step facilitates the insertion of a second guidewire into the branching blood vessel. The method is completed by inserting a second guidewire through a second lumen of the second tube and into the branching blood vessel.

Further features and advantages of the present invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art based on the teachings contained.

### BRIEF DESCRIPTION OF THE DRAWINGS

These and other features, aspects and advantages of the present invention will become better understood with reference to the following descriptions, appended claims, and accompanying drawings.

FIG. 1 is a schematic view of a guidewire loader catheter, in accordance with one embodiment of the present invention.

FIGS. 2 through 4 illustrate a method of delivering a guidewire across a bifurcation.

FIG. 5 is a schematic side view of a guidewire loader catheter, in accordance with one embodiment of the present invention.

FIG. 5A is a cross-sectional view taken along line A-A of FIG. 5.

FIG. 5B is a cross-sectional view taken along line B-B of FIG. 5.

FIG. 6 is a side schematic view of a guidewire loader catheter, in accordance with an alternative embodiment of the present invention.

FIG. 6A is a cross-sectional view taken along line A-A of FIG. 6.

FIG. 6B is a cross-sectional view taken along line B-B of FIG. 6.

FIG. 7 is a side schematic view of a guidewire loader catheter, in accordance with an alternative embodiment of the present invention.

FIG. 7A is a cross-sectional view taken along line A-A of FIG. 7.

FIG. 7B is a cross-sectional view taken along line B-B of FIG. 7.

FIG. 7C is a cross-sectional view taken along line C-C of FIG. 7.

FIG. 8 is a cross-sectional view of a surrounding sheath, in accordance with one aspect of the present invention.

FIG. 9 is a side schematic view of a guidewire loader catheter, in accordance with an alternative embodiment of the present invention.

### DETAILED DESCRIPTION OF THE INVENTION

The present invention is now described with reference to figures where like reference numbers indicate identical or functionally similar elements. Also in the figures, the left most digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and the scope of the invention.

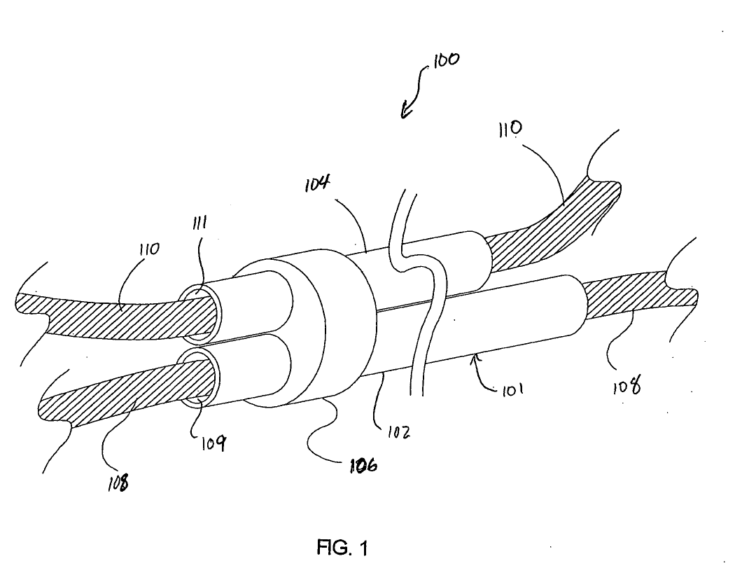

FIG. 1 shows a guidewire loader catheter 100 having a catheter shaft 101 having a first tube 102 and a second tube 104, and torque means, or a torquable handle, 106. A main vessel guidewire 108 is disposed within a first lumen 109 of the guidewire loader catheter 100. A branch vessel guidewire 110 is disposed within a second lumen 111 of the guidewire loader catheter 100.

The guidewire loader catheter 100 is constructed such that it has excellent flexibility and torquability. The guidewire loader catheter 100 can be used to position guidewires 108, 110 across branching blood vessels in a bifurcated vessel. The use of the guidewire loader catheter 100 prevents wire twisting, and facilitates cardiovascular treatments of bifurcated lesions.

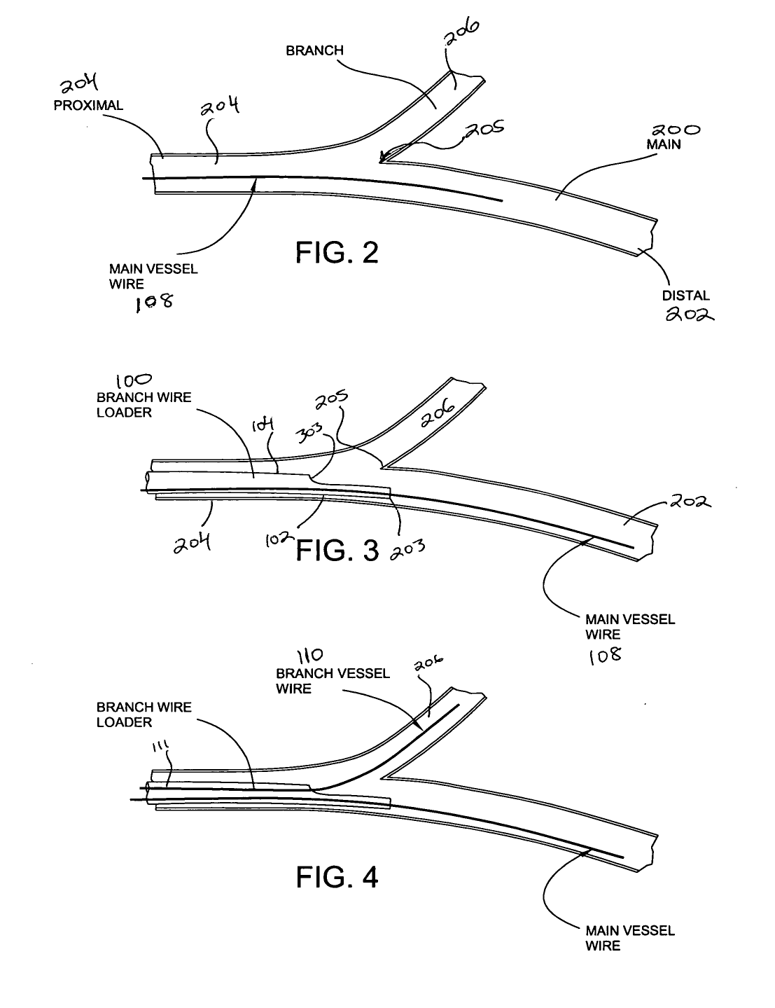

FIGS. 2 through 4 illustrate a method of delivering a guidewire across a bifurcated blood vessel. The first step of the method, as shown in FIG. 2, is to insert main vessel guidewire 108 into a main blood vessel 200, and advancing guidewire 108 until its distal end has advanced past the bifurcation 205 and into a distal section 202 of the main blood vessel 200. As shown in FIG. 3, guidewire loader catheter 100 is then tracked over main vessel guidewire 108, such that guidewire 108 (referenced in FIG. 1) is disposed within first lumen 109 of first tube 102 of catheter shaft 101. The guidewire loader catheter 100 is advanced distally until a distal end 203 of first tube 102 is distal of bifurcation 205. Second tube 104 of catheter shaft 101, however, should remain in a proximal section 204 of main blood vessel 200. At this point, a clinician may need to adjust the rotational position of guidewire loader catheter 100 to thereby align a distal end 303 of second tube 104 with a branching blood vessel 206. As shown in FIG. 4, the final step is to insert branch vessel guidewire 110 through second lumen 111 of second tube 104 of catheter shaft 101, and advance branch vessel guidewire 110 into branching blood vessel 206. As such, a clinician has successfully tracked two guidewires across bifurcation 205 and catheter 100 is then removed so further procedures may be carried out.

FIG. 5 shows one embodiment of the present invention. FIG. 5 is a schematic side view of a section of guidewire loader catheter 100 having first tube 102 and second tube 104. First tube 102 has a longitudinal length greater than a longitudinal length of second tube 104. The guidewire loader catheter 100 may be formed by a dual-lumen extrusion process. As such, the guidewire loader catheter 100 is extruded from a material such as PEBAX, polyethylene, PEEK, nylon, nylon derivatives, or polyimides. The guidewire loader catheter 100 is generally flexible and torquable, and can be used to position guidewires across a bifurcation and thereby prevent wire twisting. In one embodiment, the overall length of the guidewire loader catheter 100 is between 120 and 140 cm long.

FIG. 5A shows a cross-sectional view of the guidewire loader catheter 100 of FIG. 5 taken along line A-A. As shown, the first tube 102 has first lumen 109, and the second tube 104 has second lumen 111. In one embodiment, the cross-section of guidewire loader catheter 100 is of a shotgun-barrel shape (FIG. 8). The outer diameters of first tube 102 and second tube 104 are set to minimize the overall profile of guidewire loader catheter 100, with inner diameters still large enough to accommodate a 0.014 guidewire. In one embodiment, the overall profile of the guidewire loader catheter 100 is sized to fit within a 5-6 French guide catheter.

FIG. 5B shows a cross-sectional view of the guidewire loader catheter 100 of FIG. 5 taken along line B-B. As shown, at the point of line B-B, the second tube 104 has been cut and trimmed. As such, a distal end 203 of the first tube 102 extends distally beyond a distal end 303 of second tube 104. For example, as shown in FIG. 3, when the guidewire loader catheter 100 is inserted into blood vessel 200, the catheter can be positioned such that the first tube 102 ends distal of the bifurcation 205, and the second tube 104 ends proximal of the bifurcation 205.

FIG. 6 schematically illustrates a section of a guidewire loader catheter 600, in accordance with an alternative embodiment of the present invention. The guidewire loader catheter 600 has a catheter shaft 601 formed by a dual-lumen extrusion process. The guidewire loader catheter 600 is very flexible and torquable, and may be formed of PEBAX, polyethylene, PEEK, nylon, nylon derivatives, or polyimides. In an embodiment, metallic braids are embedded in the guidewire loader catheter 600, to improve torquability.

FIG. 6A is a cross-section taken along line A-A of FIG. 6 through a midsection of catheter shaft 601. As shown in FIG. 6A, the cross-section of the catheter shaft 601 has a circular profile. The cross-sectional profile of the catheter shaft 601 may alternatively be an oval shape. Catheter shaft 601 of guidewire loader catheter 600 includes a first lumen 609 and a second lumen 611. In one embodiment, the first and second lumen 609, 611 have inner diameters large enough to accommodate 0.014 guidewires. The overall outer diameter of catheter shaft 601 of guidewire loader catheter 600, however, is small enough so that the guidewire loader catheter 600 fits within a 5-6 French guide catheter.

In FIG. 6B, a cross-sectional view of the guidewire loader catheter 600, along line B-B of FIG. 6, shows that a distal portion of catheter shaft 601 has been removed. As such, first lumen 609 has a longer longitudinal length than that of second lumen 611. Such a configuration allows guidewire loader catheter 600 to be used in accordance with the method described above with regard to FIGS. 2-4.

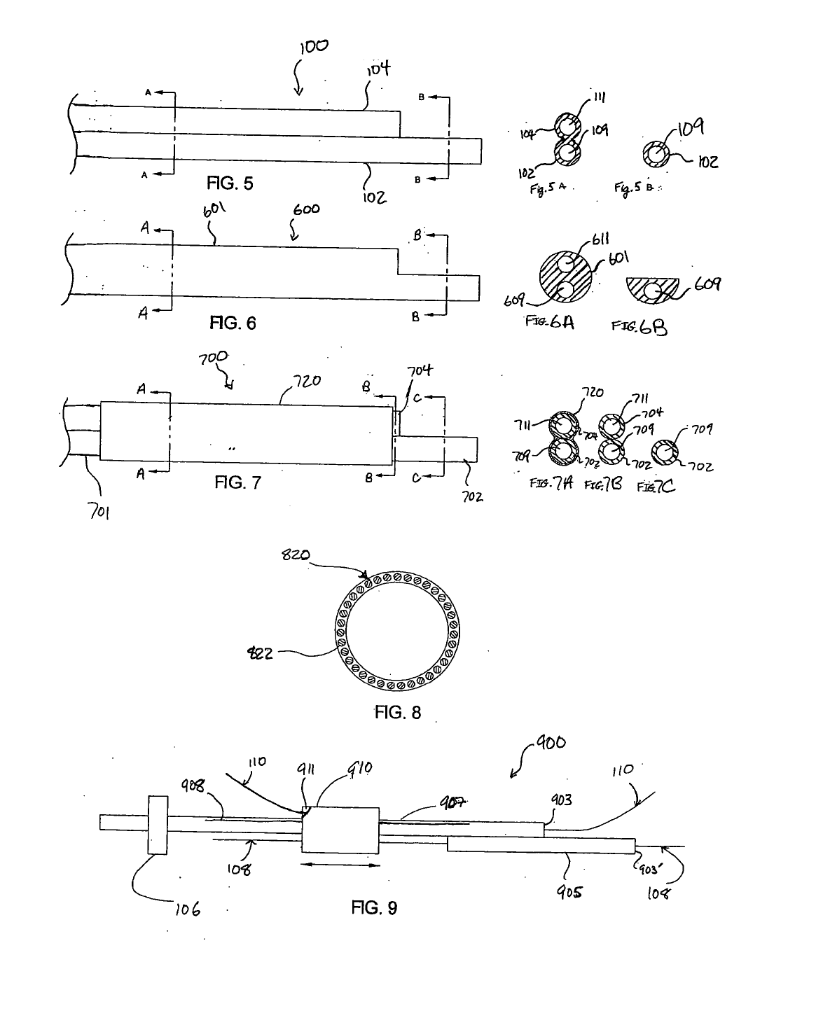

FIG. 7 schematically illustrates a section of a guidewire loader catheter 700, in accordance with an alternative embodiment of the present invention. The guidewire loader catheter 700 includes a catheter shaft 701 with a first tube 702 and a second tube 704. First tube 702 has a longitudinal length greater than a longitudinal length of second tube 704. In one embodiment, second tube 704 is tangentially attached to first tube 702, such that a lumen 711 through the second tube runs parallel to a lumen 709 through the first tube; and the first tube extends distally beyond the second tube. By “tangentially attached,” it is meant that the tubes are attached generally along a plane tangent to the outer surface of the opposite tube. The guidewire loader catheter 700 also includes a surrounding sheath 720, which envelopes a portion of catheter shaft 701, and thereby surrounds the first and second tubes 702, 704.

FIG. 7A illustrates a cross-sectional view of guidewire loader catheter 700 of FIG. 7, along a line A-A. As shown, second tube 704 is tangentially attached to first tube 702. The tubes can be attached by various methods known within the art, such as with an adhesive, tack or sonic welding, or other adhesion/fusion techniques. The surrounding sheath 720 envelopes a portion of first and second tubes 702, 704. The surrounding sheath 720 typically terminates at a point proximal of a distal end of the tubes 702, 704, as shown by FIG. 7B. FIG. 7B is a cross-sectional view of the guidewire loader catheter 700 across line B-B of FIG. 7. FIG. 7C is a cross-sectional view of the guidewire loader catheter 700 along the line C-C of FIG. 7.

FIG. 8 illustrates a cross-sectional view of a further embodiment of a surrounding sheath 820 for use in place of surrounding sheath 720 of FIG. 7. As shown, sheath 820 is formed of a polymer material having metallic braids 822 imbedded therein. In a further embodiment, catheter shafts 101 and 601 can include metallic braids, or a surrounding sheath with such, to improve pushability.

FIG. 9 is a schematic representation of an alternative embodiment, in accordance with the present invention. FIG. 9 shows a guidewire loader catheter 900 having a first catheter shaft 907 and a second catheter shaft 905. The first catheter shaft 907 includes a branch guidewire lumen extending longitudinally through first catheter shaft 907. First catheter shaft 907 further includes a guideway 908 extending longitudinally along a length of a proximal portion of a first catheter shaft 907 and extending radially from the branch guidewire lumen (not shown) to an outer surface of the proximal portion of first catheter shaft 907. Second catheter shaft 905 has a main guidewire lumen (not shown) extending longitudinally therethrough. Second catheter shaft 905 extends from a distal end 903 of first catheter shaft 907, such that a proximal end of the second catheter shaft 905 is proximal of distal end 903 of first catheter shaft 907, a distal end 903′ of the second catheter shaft 905 is distal of distal end 903 of first catheter shaft 907, and the main guidewire lumen runs parallel to the branch guidewire lumen.

The guidewire loader catheter 900 further includes a guide member 910 slidably coupled to first catheter shaft 907 and configured to provide access to the branch guidewire lumen via guideway 908. The guide member 910 also includes a guidewire exit 911. As such, a branch guidewire 110 can be inserted into guidewire exit 911 and into the guidewire lumen of first catheter shaft 907, through guide member 910. Further, guide member 910 includes a main guidewire lumen (not shown) to accommodate main guidewire 108. The guidewire loader catheter 900 further includes a torquable handle 106 to aid in positioning the guidewire loader catheter 900 within a blood vessel. A more complete description of the operation of the guide member shown in FIG. 9 can be found in U.S. Patent Application Publications: 2003-0191491 A1 published Oct. 9, 2003; 2004-0039372 A1 published Feb. 26, 2004; 2004-0059369 A1 published Mar. 25, 2004; 2004-0059291 A1 published Mar. 25, 2004; and 2004-0122363 A1 published Jun. 24, 2004; and co-pending U.S. application Ser. No. 10/722,191 filed Nov. 24, 2003, the entire disclosures of which are incorporated by reference herein. The guidewire loader catheter 900 has the added advantage that it can be used with shorter wires, as used in MX procedures, as discussed in the above referenced publications and applications.

Although a preferred embodiment of the present invention has been described, those of skill in the art will appreciate that variations and modifications may be made without departing from the spirit and scope thereof as defined by the appended claims.

What we claim is:

1. A guidewire loader catheter for delivering a guidewire across a bifurcation in a blood vessel comprising:
a catheter shaft having,
a first tube, having a first lumen extending therethrough and a first longitudinal length; and
a second tube, having a second lumen extending therethrough and a second longitudinal length, wherein the second longitudinal length is less than the first longitudinal length, and the second tube is tangentially attached to the first tube such that the second lumen runs parallel to the first lumen, wherein the first tube extends distally beyond the second tube; and
a torque means for controlling the catheter shaft, such that when the catheter shaft is inserted into a main blood vessel, the second lumen can be positioned to deliver a guidewire to a branching blood vessel.

2. The guidewire loader catheter of claim 1, further comprising a surrounding sheath, which envelops the first and second tubes of the catheter shaft.

3. The guidewire loader catheter of claim 2, wherein the surrounding sheath is formed of a polymer material having metallic braids embedded therein.

4. The guidewire loader catheter of claim 1, wherein the first and second tubes are formed of a material selected from a group consisting essentially of: PEBAX, polyethylene, PEEK, nylon, nylon derivates, and polyimides.

5. The guidewire loader catheter of claim 1, wherein the torque means includes a torquable handle attached to a proximal section of the catheter shaft.

6. The guidewire loader catheter of claim 1, wherein at least one of the first and second tubes of the catheter shaft is formed of a polymer material having metallic braids embedded therein.

7. A dual-lumen guidewire loader catheter comprising:
a tubular shaft formed of a polymer material and having
a proximal end,
a distal end,
a first lumen, with a first longitudinal length, extending from the proximal end of the tubular shaft to the distal end of the tubular shaft, and
a second lumen, with a second longitudinal length, extending from the proximal end of the tubular shaft to a point short of the distal end of the tubular shaft, wherein the second lumen is parallel to the first lumen and the second longitudinal length of the second lumen is shorter than the first longitudinal length of the first lumen, such that the tubular shaft delivers a first guidewire to a first location, in a blood vessel, through the first lumen, and a second guidewire to a second location, in a bifurcated blood vessel, through the second lumen; and,
a torquable handle attached to a proximal section of the tubular shaft to control the rotational position of the tubular shaft within the blood vessel.

8. The guidewire loader catheter of claim 7, wherein the tubular shaft is formed of a material selected from a group consisting essentially of: PEBAX, polyethylene, PEEK, nylon, nylon derivates, and polyimides.

9. The guidewire loader catheter of claim 7, wherein the tubular shaft is formed of a polymer material having metallic braids embedded therein.

10. The guidewire loader catheter of claim 7, wherein the tubular shaft further comprises a midsection having a cross-section of a generally circular shape.

11. The guidewire loader catheter of claim 7, wherein the tubular shaft further comprises a midsection having a cross-section of a generally oval shape.

12. The guidewire loader catheter of claim 7, wherein the tubular shaft further comprises a midsection having a cross-section of a generally shotgun-barrel shape.

13. A guidewire loader catheter for delivering a guidewire across a bifurcation in a blood vessel comprising:
a first catheter shaft having a proximal end, a proximal portion, a distal end, a distal portion, a branch guidewire lumen extending longitudinally through the first catheter shaft, and a guideway extending longitudinally along a length of the proximal portion of the first catheter shaft and radially from the branch guidewire lumen to an outer surface of the proximal portion of the first catheter shaft;
a second catheter shaft having a proximal end, a proximal portion, a distal end, a distal portion, a main guidewire lumen extending longitudinally through said second catheter shaft, and wherein the second catheter shaft extends from the distal portion of the first catheter shaft such that the proximal end of the second catheter shaft is proximal of the distal end of the first catheter shaft, the distal end of the second catheter shaft is distal of the distal end of the first catheter shaft, and the main guidewire lumen runs parallel to the branch guidewire lumen; and
a guide member slidably coupled to the first catheter shaft and configured to provide access to the branch guidewire lumen via the guideway.

14. The guidewire loader catheter of claim 13, further including a torquable handle disposed about the proximal section of the first catheter shaft, proximal of the guide member.

15. The guidewire loader catheter of claim 13, wherein the guide member further includes a main guidewire lumen, aligned with the main guidewire lumen of the second catheter shaft, such that a guidewire may be inserted through the main guidewire lumen of the second catheter shaft and through the main guidewire lumen of the guide member.

16. The guidewire loader catheter of claim 13, wherein the first catheter shaft is formed of a material selected from a group consisting essentially of: PEBAX, polyethylene, PEEK, nylon, nylon derivates, and polyimides.

17. The guidewire loader catheter of claim 13, wherein the second catheter shaft is formed of a material selected from a group consisting essentially of: PEBAX, polyethylene, PEEK, nylon, nylon derivates, and polyimides.

18. The guidewire loader catheter of claim 13, wherein the first catheter shaft is formed of a polymer material having metallic braids embedded therein.

19. The guidewire loader catheter of claim 13, wherein the second catheter shaft is formed of a polymer material having metallic braids embedded therein.

20. A method of delivering a guidewire across a bifurcation comprising:
providing a guidewire loader catheter comprising: a first tube, having a first lumen extending therethrough and a first longitudinal length, a second tube, having a second lumen extending therethrough and a second longitudinal length, wherein the second longitudinal length is less than the first longitudinal length, and the second tube is tangentially attached to the first tube such that the second lumen runs parallel to the first lumen, wherein the first tube extends distally beyond the second tube, and a torque means for controlling the rotational position of the guidewire loader catheter;
inserting a first guidewire through a first blood vessel to a point distal of a bifurcation between the first blood vessel and a branching blood vessel;
tracking the guidewire loader catheter over the first guidewire, such that the first guidewire is disposed within the first lumen of the first tube;
advancing the guidewire loader catheter until a distal end of the first tube extends beyond the bifurcation and a distal end of the second tube remains proximal of the bifurcation; and
inserting a second guidewire through the second lumen of the second tube and into the branching blood vessel.

21. The method of claim 20, further comprising an adjustment step prior to the inserting step, wherein the adjustment step comprises adjusting the rotational position of the distal end of the second tube with respect to the branching blood vessel, using the torque means, to thereby facilitate the insertion of the second guidewire into the branching blood vessel.

22. The method of claim 20, further comprising providing a torque means, wherein the torque means is a torquable handle attached to a proximal section of the guidewire loader catheter.

23. A method of delivering a guidewire across a bifurcation comprising:
providing a dual-lumen guidewire loader catheter having a first lumen, with a first longitudinal length, extending from a proximal end to a distal end of the guidewire loader catheter, a second lumen, with a second longitudinal length, extending from the proximal end to a point short of the distal end of the guidewire loader catheter, wherein the second lumen is parallel to the first lumen and the longitudinal length of the first lumen is longer than the longitudinal length of the second lumen, and a torquable handle attached to a proximal section of the guidewire loader catheter and configured to control the rotational position of the guidewire loader catheter within a blood vessel;
inserting a first guidewire through a first blood vessel to a point distal of a bifurcation between the first blood vessel and a branching blood vessel;
tracking the guidewire loader catheter over the first guidewire, such that the first guidewire is disposed within the first lumen of the guidewire loader catheter;
advancing the guidewire loader catheter until the distal end of the guidewire loader catheter extends distally beyond the bifurcation and a distal end of the second lumen remains proximal of the bifurcation; and
inserting a second guidewire through the second lumen of the guidewire loader catheter and into the branching blood vessel.

24. The method of claim 23, further comprising an adjustment step prior to the inserting step, wherein the adjustment step comprises adjusting the rotational position of the guidewire loader catheter with respect to the branching blood vessel, using the torquable handle, to thereby facilitate the insertion of the second guidewire into the branching blood vessel.
